(19) United States
(12) Patent Application Publication
(10) Pub. No.: US 2016/0136169 A1
(43) Pub. Date: May 19, 2016

(54) SYSTEMS AND METHODS FOR DELIVERY OF TETRAHYDROBIOPTERIN AND RELATED COMPOUNDS

(71) Applicant: Transdermal Biotechnology, Inc., Meriden, CT (US)

(72) Inventor: Nicholas V. Perricone, Madison, CT (US)

(73) Assignee: Transdermal Biotechnology, Inc., Meriden, CT (US)

(21) Appl. No.: 14/943,301

(22) Filed: Nov. 17, 2015

Related U.S. Application Data
(63) Continuation-in-part of application No. 14/492,227, filed on Sep. 22, 2014, now Pat. No. 9,295,638.
Application No. 14/492,227 is a continuation of application No. 13/801,231, filed on Mar. 13, 2013, now Pat. No. 8,871,257.
Application No. 13/801,231 is a continuation-in-part of application No. 13/623,018, filed on Sep. 19, 2012, now abandoned.
(60) Provisional application No. 62/081,175, filed on Nov. 18, 2014.

(51) Int. Cl. A61K 31/519; A61K 33/00; A61K 9/00; A61K 9/06

(52) U.S. Cl. CPC A61K 31/519; A61K 9/06; A61K 33/00; A61K 9/0014

(57) ABSTRACT

The present invention generally relates to compositions and methods for the delivery of tetrahydrobiopterin and/or related compounds through the skin. In general, tetrahydrobiopterin may react with oxygen, and various formulations discussed herein may prevent or reduce the ability of tetrahydrobiopterin to react with oxygen. For example, in some aspects, the tetrahydrobiopterin may be contained with a composition comprising a lecithin, such as phosphatidylcholine. In certain embodiments, the lecithin is present in liposomes, micelles, liquid crystals, or other configurations. The composition can take the form of a gel, a cream, a lotion, an ointment, a solution, a solid “stick,” etc., that can be rubbed or sprayed onto the skin. Other aspects of the present invention are generally directed to methods of making or using such compositions, methods of promoting such compositions, kits including such compositions, or the like.

### RELATED APPLICATIONS

This application is a continuation-in-part of U.S. patent application Ser. No. 14/492,227, filed Sep. 22, 2014, entitled “Prevention and Treatment of Cardiovascular Diseases Using Systems and Methods for Transdermal Nitric Oxide Delivery,” which is a continuation of U.S. patent application Ser. No. 13/801,231, filed Mar. 13, 2013, entitled “Prevention and Treatment of Cardiovascular Diseases Using Systems and Methods for Transdermal Nitric Oxide Delivery,” which is a continuation-in-part of U.S. patent application Ser. No. 13/623,018, filed Sep. 19, 2012, entitled “Prevention and Treatment of Cardiovascular Diseases Using Systems and Methods for Transdermal Nitric Oxide Delivery.” This application also claims the benefit of U.S. Provisional Patent Application Ser. No. 62/081,175, filed Nov. 18, 2014, entitled “Systems and Methods for Delivery of Tetrahydrobiopterin and Related Compounds.” Each of these is incorporated herein by reference in its entirety.

### FIELD

The present invention generally relates to compositions and methods for the delivery of tetrahydrobiopterin and/or related compounds through the skin.

### BACKGROUND

Tetrahydrobiopterin (or sapropterin) is a naturally occurring essential cofactor of several aromatic amino acid hydroxylase enzymes. It is believed to be involved in the degradation of the amino acid phenylalanine and in the biosynthesis of the neurotransmitters serotonin (5-hydroxytryptamine, 5-HT), melatonin, dopamine, norepinephrine (noradrenaline), epinephrine (adrenaline). It is also a cofactor for the production of nitric oxide (NO) by nitric oxide synthases, and thus may play a role in regulating NO in the body.

However, tetrahydrobiopterin is relatively reactive, and may degrade upon contact with oxygen or air. It is often stored in oxygen-free or degassed solutions, or it may stored in dry form to prevent oxygen degradation. Within a liquid, efforts must be made to maintain an oxygen-free environment in order to prevent the degradation of tetrahydrobiopterin prior to use. In commercial applications, tetrahydrobiopterin is often shipped dry and must be reconstituted in water or saline immediately prior to oral administration. Due to these properties, it has been difficult to create a topical form of tetrahydrobiopterin, since liquid formulations containing tetrahydrobiopterin will often contain dissolved oxygen as well (e.g., once reaching equilibrium with the surrounding air), causing the tetrahydrobiopterin to break down relatively rapidly, often within minutes.

### SUMMARY

The present invention generally relates to compositions and methods for the delivery of tetrahydrobiopterin and/or related compounds through the skin. The subject matter of the present invention involves, in some cases, interrelated products, alternative solutions to a particular problem, and/or a plurality of different uses of one or more systems and/or articles.

In one aspect, the present invention is generally directed to an article for transdermal delivery. According to one set of embodiments, the article comprises a composition comprising a carrier and lecithin, the lecithin containing tetrahydrobiopterin and/or a salt thereof. In some embodiments, the composition further comprises no more than about 250 ppm of water by weight of the composition.

The article, in another set of embodiments, includes a composition comprising a carrier and lecithin, the lecithin containing tetrahydrobiopterin and/or a salt thereof. In certain instances, the composition is stable at room temperature.

The article, according to another set of embodiments, comprises a composition comprising a carrier and lecithin, the lecithin containing dihydrobiopterin and/or salt thereof. In some cases, the composition further comprises no more than about 250 ppm of water by weight of the composition.

The article, in yet another set of embodiments, comprises a composition comprising a carrier and lecithin, the lecithin containing dihydrobiopterin and/or a salt thereof. In certain embodiments, the composition is stable at room temperature.

In still another set of embodiments, the article comprises a composition comprising a carrier and lecithin, the lecithin containing quinonoid dihydrobiopterin and/or salt thereof. In some embodiments, the composition further comprises no more than about 250 ppm of water by weight of the composition.

The article, in another set of embodiments, includes a composition comprising a carrier and lecithin, the lecithin containing quinonoid dihydrobiopterin and/or a salt thereof. In some cases, the composition is stable at room temperature.

The present invention, in another aspect, is directed to a method. In one set of embodiments, the method includes administering, to a subject, a composition comprising a carrier and lecithin, the lecithin containing tetrahydrobiopterin and/or a salt thereof. In some cases, the composition further comprises no more than about 250 ppm of water by weight of the composition.

The method, in another set of embodiments, includes administering, to a subject, a composition comprising a carrier and lecithin, the lecithin containing tetrahydrobiopterin and/or a salt thereof. In certain instances, the composition is stable at room temperature.

According to yet another set of embodiments, the method includes administering, to a subject, a composition comprising a carrier and lecithin, the lecithin containing dihydrobiopterin and/or a salt thereof. In some cases, the composition further comprises no more than about 250 ppm of water by weight of the composition.

In still another set of embodiments, the method includes administering, to a subject, a composition comprising a carrier and lecithin, the lecithin containing dihydrobiopterin and/or a salt thereof. In some cases, the composition is stable at room temperature.

The method, in yet another set of embodiments, comprises administering, to a subject, a composition comprising a carrier and lecithin, the lecithin containing quinonoid dihydrobiopterin and/or a salt thereof. In certain embodiments, the composition further comprises no more than about 250 ppm of water by weight of the composition.

In accordance with another set of embodiments, the method includes administering, to a subject, a composition comprising a carrier and lecithin, the lecithin containing quinonoid dihydrobiopterin and/or a salt thereof. In some cases, the composition is stable at room temperature.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention encompasses methods of making one or more of the embodiments described herein, for example, a composition comprising tetrahydrobiopterin. In still another aspect, the present invention encompasses methods of using one or more of the embodiments described herein, for example, a composition comprising tetrahydrobiopterin.

Other advantages and novel features of the present invention will become apparent from the following detailed description of various non-limiting embodiments of the invention. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### DETAILED DESCRIPTION

The present invention generally relates to compositions and methods for the delivery of tetrahydrobiopterin and/or related compounds through the skin. In general, tetrahydrobiopterin may react with oxygen, and various formulations discussed herein may prevent or reduce the ability of tetrahydrobiopterin to react with oxygen. For example, in some aspects, the tetrahydrobiopterin may be contained with a composition comprising a lecithin, such as phosphatidylcholine. In certain embodiments, the lecithin is present in liposomes, micelles, liquid crystals, or other configurations. The composition can take the form of a gel, a cream, a lotion, an ointment, a solution, a solid “stick,” etc., that can be rubbed or sprayed onto the skin. Other aspects of the present invention are generally directed to methods of making or using such compositions, methods of promoting such compositions, kits including such compositions, or the like.

In certain aspects, the present invention is generally directed to compositions comprising tetrahydrobiopterin, including salts thereof, and/or related compounds, for application to the skin of a subject, e.g., a human subject. In some embodiments, the composition is generally directed to a cream, or other formulation, that contains a lecithin, such as phosphatidylcholine. The lecithin may be useful in preventing tetrahydrobiopterin from degrading in the presence of oxygen and/or in delivering the tetrahydrobiopterin across the skin of a subject, e.g., to the bloodstream. The composition may be applied to the skin of a subject, such as a human subject, to treat a variety of diseases or conditions. For example, in one set of embodiments, the composition may be used to treat a subject having phenylketonuria or tetrahydrobiopterin deficiencies. In addition, in some cases, the composition may be used to treat a subject having or at risk for cardiovascular disease, e.g., due to its effects on nitric oxide synthase and nitric oxide levels. Other examples of treatment of various diseases or conditions are discussed in additional detail below.

As mentioned, certain aspects of the invention are generally directed to compositions comprising tetrahydrobiopterin and/or related compounds, e.g., for topical or transdermal delivery. However, it should be understood that although much of the description herein recites tetrahydrobiopterin, this is for ease of presentation, and that in other embodiments of the invention, other compounds may be used in addition to or instead of tetrahydrobiopterin. Non-limiting examples of compounds closely related to tetrahydrobiopterin include dihydrobiopterin, quinonoid dihydrobiopterin, 7,8-dihydrobiopterin, or the like.

Tetrahydrobiopterin generally has the following structure:

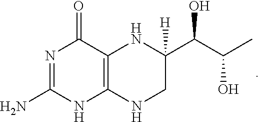 [SMILES: C[C@H](O)[C@H](O)[C@H]1CNc2[nH]c(N)nc(=O)c2N1]

The tetrahydrobiopterin may be present as the structure provided above, and/or in a salt form, e.g., where the above structure is ionized and is in the presence of one or more suitable counterions. For example, tetrahydrobiopterin may be present as a free base, free acid, or a pharmaceutically acceptable salt such as tetrahydrobiopterin dichloride. Other forms of tetrahydrobiopterin are also possible, e.g., as a precursor, derivative, polymorph, hydrate, enantiomer, tautomer, racemic mixture, analog, or the like. Tetrahydrobiopterin is also sold commercially in various forms, for instance, under the brand name Kuvan®.

Suitable precursors of tetrahydrobiopterin that may be employed in certain embodiments of the invention include, but are not limited to, dihydroneopterin triphosphate, biopterin, sepiapterin, 7,8-dihydrobiopterin and the like, or mixtures thereof. Suitable derivatives of tetrahydrobiopterin that may be employed include, but are not limited to, N2-methyl-H4-biopterin, N5-methyl-H4-biopterin, N5-formyl-H4-biopterin, N5-acetyl-H4-biopterin, 1′,2′-diacetyl-5,6,7,8-tetrahydrobiopterin, 6-methyl-5,6,7,8-tetrahydropterin, 6-hydroxymethyl-5,6,7,8-tetrahydropterin, 6-phenyl-5,6,7,8-tetrahydropterin, hydrazine derivatives of tetrahydrobiopterin, 2-N-stearoyl-1′,2′-di-O-acetyl-L-biopterin, L-tetrahydrobiopterin, tetrahydrofuranylpyrimidine derivative, 7,8-dihydrobiopterin, lipoic acid derivative of tetrahydrobiopterin such as dihydrolipoic acid and the like, or mixtures thereof. Suitable analogs of tetrahydrobiopterin that may be employed include, but are not limited to, 6-methoxymethyl-tetrahydropterin, pteridine, neopterin, biopterin, 7,8-dihydrobiopterin, 6-methyltetrahydropterin, 6-substituted tetrahydropterin, 6R-L-erythro-tetrahydrobiopterin, sepiapterin, 6,7-dimethyltetrahydropterin, 6-methyl biopterin, 7-tetrahydrobiopterin and the like, or mixtures thereof.

A compound related to tetrahydrobiopterin is dihydrobiopterin, which differs from tetrahydrobiopterin mainly by two hydrogen atoms. Dihydrobiopterin generally has the following structure:

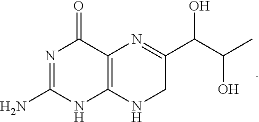 [SMILES: CC(O)C(O)C1=Nc2c([nH]c(N)nc2=O)NC1]

Another related compound is quinonoid dihydrobiopterin, which generally has the following structure:

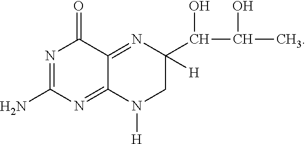 [SMILES: CC(O)C(O)C1CNC2=NC(N)=NC(=O)C2=N1]

Yet another related compound is 7,8-dihydrobiopterin, which generally has the following structure:

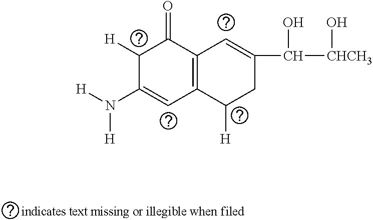

The above compounds have structures similar to tetrahydrobiopterin, and may be present in certain embodiments in the structures provided above, and/or in a salt form, e.g., where the above structures are ionized and are in the presence of one or more counterions. For example, these compounds may be present as a free base, free acid, or pharmaceutically acceptable salt, e.g., as in dihydrobiopterin dichloride. Other forms are also possible, e.g., as a precursor, derivative, polymorph, hydrate, enantiomer, tautomer, racemic mixture, analog, or the like. These compounds may also be readily obtained commercially.

Accordingly, in the descriptions of formulations, etc. that follow that describe tetrahydrobiopterin only, it should be understood that this is by way of example and ease of presentation, and that in any of the following descriptions, other embodiments involving any of the above compounds such as dihydrobiopterin, quinonoid dihydrobiopterin, or 7,8-dihydrobiopterin, or other related compounds, are also possible. In addition, more of one of these may also be present in some embodiments, e.g., a composition may comprise tetrahydrobiopterin and dihydrobiopterin, tetrahydrobiopterin and quinonoid dihydrobiopterin, tetrahydrobiopterin and 7,8-dihydrobiopterin, dihydrobiopterin and quinonoid dihydrobiopterin, etc.

As discussed, suitable pharmaceutically acceptable salts, such as, but not limited to, acid or base addition salts may be employed in certain embodiments. Suitable pharmaceutically acceptable base addition salts of tetrahydrobiopterin or a related compound such as dihydrobiopterin, quinonoid dihydrobiopterin, 7,8-dihydrobiopterin, etc. may be formed with metals or amines, such as, but not limited to, alkali and alkaline earth metals or organic amines. Pharmaceutically acceptable salts may also be prepared with a pharmaceutically acceptable cation such as, but not limited to, alkaline, alkaline earth, ammonium and quaternary ammonium cations. Suitable metals include, but are not limited to sodium, potassium, magnesium, ammonium, calcium, or ferric, and the like. Suitable amines include, but are not limited to isopropylamine, trimethylamine, histidine, N,N′-dibenzylethylenediamine, chloroprocaine, choline, diethanolamine, dicyclohexylamine, ethylenediamine, N-methylglucamine, and procaine. Suitable pharmaceutically acceptable acid addition salts include, but are not limited to, inorganic or organic acid salts. Examples of suitable acid salts include, but are not limited to, hydrochlorides, acetates, citrates, salicylates, nitrates, phosphates. Other suitable pharmaceutically acceptable salts include, for example, acetic, citric, oxalic, tartaric, or mandelic acids, hydrochloric acid, hydrobromic acid, sulfuric acid or phosphoric acid; organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and amino acids, such as the 20 alpha amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoc acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. For instance, in various embodiments, the tetrahydrobiopterin or related compounds such as dihydrobiopterin, quinonoid dihydrobiopterin, 7,8-dihydrobiopterin, etc. may be employed in a composition in the form of a dichloride or dihydrochloride salt.

In various embodiments, the tetrahydrobiopterin employed in a composition is sapropterin. In yet another embodiment, tetrahydrobiopterin employed in a composition is (6R)-L-erythro-5,6,7,8-tetrahydrobiopterin dihydrochloride or (6R)-2-amino-6-[(1R,2S)-1,2-dihydroxypropyl]-5,6,7,8-tetrahydro-4(1H)-pteridinone dihydrochloride or sapropterin dihydrochloride.

Any suitable amount of tetrahydrobiopterin and/or a related compound such as dihydrobiopterin, quinonoid dihydrobiopterin, 7,8-dihydrobiopterin, etc. may be present within a composition prepared as described herein. For example, at least about 0.3 wt %, at least about 0.5 wt %, at least about 0.7 wt %, at least about 1 wt %, at least about 1.5 wt %, at least about 2 wt %, at least about 2.5 wt %, at least about 3 wt %, at least about 5 wt % at least about 10 wt %, at least about 20 wt %, at least about 30 wt %, at least about 40 wt %, at least about 50 wt %, at least about 60 wt %, at least about 70 wt %, at least about 80 wt %, at least about 90 wt %, at least about 100 wt %, at least about 110 wt %, or at least about 120 wt % of the composition can be tetrahydrobiopterin and/or a related compound, where the basis of the weight percentage is the weight of the composition before the tetrahydrobiopterin and/or related compound is added. Combinations of any of these are also possible. For example, the tetrahydrobiopterin and/or related compound may be present at between 70 wt % and about 120 wt % of the composition.

In some embodiments, the tetrahydrobiopterin and/or related compounds such as dihydrobiopterin, quinonoid dihydrobiopterin, 7,8-dihydrobiopterin, etc. may be present at a concentration of at least about 400 mg/kg, at least about 450 mg/kg, at least about 500 mg/kg, at least about 550 mg/kg, at least about 570 mg/kg, at least about 600 mg/kg, at least about 650 mg/kg, at least about 700 mg/kg, at least about 750 mg/kg, at least about 800 mg/kg, at least about 850 mg/kg, at least about 950 mg/kg, or at least about 1000 mg/kg of the composition, where the mass that is determined is that of the final composition, e.g., including the tetrahydrobiopterin and/or related compound. In certain cases, the tetrahydrobiopterin and/or related compounds may be present at a concentration of no more than about 2000 mg/kg, no more than about 1500 mg/kg, no more than about 1000 mg/kg, no more than about 960 mg/kg, no more than about 900 mg/kg, no more than about 800 mg/kg, no more than about 700 mg/kg, or no more than about 600 mg/kg. Combinations of any of these are also possible. For example, the tetrahydrobiopterin and/or related compounds may be present at a concentration of between about 570 mg/kg and about 960 mg/kg.

In some embodiments, tetrahydrobiopterin and/or a related compound such as dihydrobiopterin, quinonoid dihydrobiopterin, 7,8-dihydrobiopterin, etc. is present at a concentration (e.g., on a per-mass basis) of at least about 100 ppm, at least about 200 ppm, at least about 300 ppm, at least about 400 ppm, at least about 500 ppm, at least about 600 ppm, at least about 700 ppm, at least about 800 ppm, at least about 900 ppm, at least about 1000 ppm, at least about 1100 ppm, at least about 1200 ppm, at least about 1300 ppm, at least about 1400 ppm, at least about 1500 ppm, at least about 1600 ppm, at least about 1700 ppm, at least about 1800 ppm, at least about 1900 ppm, at least about 2000 ppm, at least about 2500 ppm, at least about 3000 ppm, at least about 3500 ppm, at least about 4000 ppm, at least about 4500 ppm, at least about 5000 ppm, at least about 6000 ppm, at least about 7000 ppm, at least about 8000 ppm, at least about 9000 ppm, or at least about 10000 ppm of the composition. In other embodiments, the tetrahydrobiopterin and/or related compound is present at a concentration of no more than about 11000 ppm, no more than about 10000 ppm, no more than about 9000 ppm, no more than about 8000 ppm, no more than about 7000 ppm, no more than about 6000 ppm, no more than about 5000 ppm, no more than about 4500 ppm, no more than about 4000 ppm, no more than about 3500 ppm, no more than about 3000 ppm, no more than about 2500 ppm, no more than about 2000 ppm, no more than about 1900 ppm, no more than about 1800 ppm, no more than about 1700 ppm, no more than about 1600 ppm, no more than about 1500 ppm, no more than about 1400 ppm, no more than about 1300 ppm, no more than about 1200 ppm, no more than about 1100 ppm, no more than about 1000 ppm, no more than about 900 ppm, no more than about 800 ppm, no more than about 700 ppm, no more than about 600 ppm, no more than about 500 ppm, no more than about 400 ppm, or no more than about 300 ppm of the composition. Combinations of any of these are also possible. For example, in some embodiments, the tetrahydrobiopterin and/or related compound is present at a concentration of between about 400 and about 900 ppm.

In addition, in some cases, the composition may include tetrahydrobiopterin and/or a related compound, and nitric oxide (NO). However, it should be understood that in some cases, the composition is free from any nitric oxide. If present, the NO may be present in gaseous or molecular form. As mentioned above, tetrahydrobiopterin is a cofactor for the enzyme nitric oxide synthase, and thus, in some embodiments, both NO and tetrahydrobiopterin (or a related compound, e.g., which may be converted into tetrahydrobiopterin and/or provide biological effects similar to tetrahydrobiopterin) may be applied to the skin of a subject to facilitate the treatment of diseases or conditions that can be treated by NO, e.g., topically or transdermally. Examples include, but are not limited to treatment of acne, infection, dermatoses, inflammatory disease, atherosclerosis, hypertension, sickle-cell anemia, cardiovascular disease, Alzheimer's disease, Parkinson's disease, neural disease, neuromuscular disease, osteoporosis, neuropathy, or cancer. Additional examples and nitric oxide are both further discussed in additional detail below.

In certain aspects of the invention, tetrahydrobiopterin and/or a related compound such as dihydrobiopterin, quinonoid dihydrobiopterin, 7,8-dihydrobiopterin, etc. may be contained within a composition that is to be applied to the skin, e.g., such that tetrahydrobiopterin or the related compound may be administered for topical or transdermal delivery. In some cases, the composition is a cream, although other formulations are also possible in some instances, e.g., a liquid, a gel, a lotion, an ointment, a solid “stick,” or the like, such as is discussed herein.

In certain embodiments of the present invention, tetrahydrobiopterin and/or a related compound such as dihydrobiopterin, quinonoid dihydrobiopterin, 7,8-dihydrobiopterin, etc. may be contained within certain compositions comprising lecithin or phosphatidylcholine. In one set of embodiments, the composition may comprise liquid crystal multilamellar phosphatidylcholine. In some cases, the compositions are relatively free of oxygen (O₂) or water. Without wishing to be bound by any theory, it is believed that such compositions may serve to inhibit or reduce reaction of tetrahydrobiopterin (or a related compound) with oxygen (e.g., in the air, or dissolved in water, etc.). Thus, in some cases, the compositions may be stable, and/or can be stored for periods of time with little or no loss or reaction of the tetrahydrobiopterin contained therein. In some cases, stability of the composition can be achieved at room temperature (about 25° C.), and/or at other storage temperatures such as those described herein.

In one set of embodiments, the composition comprises a first phase comprising a lecithin such as phosphatidylcholine, which may be present within a second phase comprising an emulsifier, such as is discussed herein. Other components, for example, transdermal penetration enhancers, adjuvants, surfactants, lubricants, etc. can also be present in certain cases.

The compositions of the invention comprise, in certain embodiments, a phase comprising phosphatidylcholine and/or other lecithins in which tetrahydrobiopterin and/or a related compound such as dihydrobiopterin, quinonoid dihydrobiopterin, 7,8-dihydrobiopterin, etc. may be contained within, e.g., to reduce the ability of oxygen (e.g., from the air) to react with the tetrahydrobiopterin (or related compound). In some cases, the phosphatidylcholine or lecithin may be contained within a second phase, for example, comprising an emulsifier, which may cause the phosphatidylcholine or lecithin to form vesicles, e.g., micelles or liposomes. The phosphatidylcholine or lecithin composition can be unilamellar or multilamellar in some embodiments. However, in some instances, the presence of the second phase causes the phosphatidylcholine or lecithin to form a liquid crystal arrangement, rather than a vesicular or liposomal arrangement.

Without wishing to be bound by any theory, it is believed that the phosphatidylcholine or other lecithin may be used to surround the tetrahydrobiopterin (or related compound), thereby reducing access to oxygen. In some cases, the tetrahydrobiopterin (or related compound) may be contained within water or other aqueous environment within the composition (e.g., within vesicles such as liposomes or an emulsion within the composition, etc.), although in some embodiments, little or no water is used and the tetrahydrobiopterin (or related compound) is directly contained within the phosphatidylcholine or other lecithin within the composition.

For example, in certain embodiments of the invention, the composition, or at least a phase of the composition comprising tetrahydrobiopterin and/or a related compound such as dihydrobiopterin, quinonoid dihydrobiopterin, 7,8-dihydrobiopterin, etc. is substantially free of water, e.g., comprising no more than about 10 wt %, no more than about 3 wt %, no more than about 1 wt %, no more than about 0.3 wt %, or no more than about 0.1 wt % water (i.e., relative to the weight of the overall composition). The composition may also have no more than about 1,000 ppm, no more than about 750 ppm, no more than about 500 ppm, no more than about 400 ppm, no more than about 300 ppm, no more than about 250 ppm, no more than about 200 ppm, no more than about 150 ppm, no more than about 100 ppm, no more than about 50 ppm, no more than about 25 ppm, or no more than about 10 ppm of water (by weight). In certain embodiments, no detectable water may be present in the composition, or at least within a phase of the composition comprising the tetrahydrobiopterin and/or related compound. Any suitable technique can be used for determining the amount of water present in the composition, for example, Karl-Fisher titration. In some cases, the composition may also be free of any liquids that typically contain water, e.g., physiological buffers, bodily fluids, saline, blood, or the like.

In addition, in some embodiments, the composition, or at least a phase of the composition comprising tetrahydrobiopterin and/or a related compound such as dihydrobiopterin, quinonoid dihydrobiopterin, 7,8-dihydrobiopterin, etc. is substantially free of gaseous oxygen (O₂). For instance, the composition may also have no more than about 1,000 ppm, no more than about 750 ppm, no more than about 500 ppm, no more than about 400 ppm, no more than about 300 ppm, no more than about 250 ppm, no more than about 200 ppm, no more than about 150 ppm, no more than about 100 ppm, no more than about 50 ppm, no more than about 25 ppm, or no more than about 10 ppm of oxygen (by weight).

Phosphatidylcholine (herein abbreviated “PC”) is a basic component of cell membrane bilayers and the main phospholipid circulating in the plasma of blood. Phosphatidylcholine typically has a phospholipid structure with a choline head group and a glycerophosphoric acid tail group. The tail group can be saturated or unsaturated. More than one tail group may be present in the phosphatidylcholine in some cases, and the tail groups may be the same or different. Specific non-limiting examples of phosphatidylcholines that could be used include one or a mixture of stearic, palmitic, margaric, and/or oleic acid diglycerides linked to a choline ester head group.

Phosphatidylcholines are a member of a class of compounds called lecithins. Typically, a lecithin is a composed of phosphoric acid, choline, fatty acids, glycerol, glycolipids, triglycerides, and/or phospholipids. In some cases, other lecithins may be used, in addition to or instead of a phosphatidylcholine. Non-limiting examples of other lecithins include phosphatidylethanolamine, phosphatidylinositol, or phosphatidic acid. Many commercial lecithin products are available, such as, for example, Lecithol®, Vitellin®, Kelecin®, and Granulestin®. Lecithin is widely used in the food industry. In some embodiments, certain compositions of the invention can contain synthetic or natural lecithin, or mixtures thereof. Natural preparations are used in some cases because they exhibit desirable physical characteristics, and/or may be economical or nontoxic. However, in other embodiments, non-natural preparations are used, or the composition can include both natural and non-natural preparations.

Any suitable amount of phosphatidylcholine or lecithin may be present within the composition. For example, at least about 0.25 wt %, at least about 0.5 wt %, at least about 1 wt %, at least about 2 wt %, at least about 3 wt %, at least about 5 wt %, at least about 8 wt %, at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the entire composition can be a phosphatidylcholine or a lecithin. In some cases, the phosphatidylcholine or lecithin may be present at a concentration of no more than about 95 wt %, no more than about 90 wt %, no more than about 80 wt %, no more than about 70 wt %, no more than about 65 wt %, no more than about 60 wt %, no more than about 50 wt %, no more than about 40 wt %, no more than about 30 wt %, no more than about 20 wt %, or no more than about 10%. Combinations of any of these are also possible. For instance, the phosphatidylcholine or lecithin may be present at between about 8 wt % and about 65 wt %, or between about 0 wt % and about 10 wt %, etc. One or more than one type of phosphatidylcholine or lecithin may be present.

In some aspects, the formulation comprises a phosphatidylcholine, e.g., any of those described herein. The composition can include any suitable amount of phosphatidylcholine, for example, at least about 1 wt %, at least about 3 wt %, at least about 5 wt %, at least about 10 wt %, at least about 20 wt %, at least about 30 wt %, at least about 40 wt %, at least about 50 wt %, at least about 60 wt %, at least about 70 wt %, at least about 80 wt %, at least about 90 wt % etc. In some cases, no more than about 90 wt %, no more than about 80 wt %, no more than about 70 wt %, no more than about 60 wt %, no more than about 50 wt %, no more than about 40 wt %, no more than about 30 wt %, no more than about 20 wt %, no more than about 10 wt %, or no more than about 5 wt % of the composition is phosphatidylcholine. Combinations of any of these are also possible. For example, the composition may be between about 0 wt % and about 10 wt % surfactant. The composition may include one or more than one phosphatidylcholine. One non-limiting example of a phosphatidylcholine is Phospholipon-90G (American Lecithin Company).

Some compositions may contain polyenylphosphatidylcholine (herein abbreviated “PPC”). In some cases, PPC can be used to enhance epidermal penetration. The term “polyenylphosphatidylcholine,” as used herein, means any phosphatidylcholine bearing two fatty acid moieties, wherein at least one of the two fatty acids is an unsaturated fatty acid with at least two double bonds in its structure, such as linoleic acid.

Certain types of soybean lecithin and soybean fractions, for example, can contain higher levels of polyenylphosphatidylcholine, with dilinoleoylphosphatidylcholine (18:2-18:2 phosphatidylcholine) as the most abundant phosphatidylcholine species therein, than conventional food grade lecithin. Such lecithins may be useful in formulating certain delivery compositions. In some embodiments, conventional soybean lecithin may be enriched with polyenylphosphatidylcholine, for instance, by adding soybean extracts containing high levels of polyenylphosphatidylcholine. As used herein, this type of phosphatidylcholine is called “polyenylphosphatidylcholine-enriched” phosphatidylcholine (hereinafter referred to as PPC-enriched phosphatidylcholine), even where the term encompasses lecithin obtained from natural sources exhibiting polyenylphosphatidylcholine levels higher than ordinary soybean varieties. These products are commercially available, for example, from American Lecithin Company, Rhone-Poulenc and other lecithin vendors. American Lecithin Company markets its products with a “U” designation, indicating high levels of unsaturation; Rhone-Poulenc's product is a soybean extract containing about 42% dilinoleoylphosphatidylcholine and about 24% palmitoyllinoleylphosphatidylcholine (16:0 to 18:2 of PC) as the major phosphatidylcholine components. Another example of a suitable polyenylphosphatidylcholine is NAT 8729 (also commercially available from vendors such as Rhone-Poulenc and American Lecithin Company).

Any suitable amount of polyenylphosphatidylcholine may be present within the composition. For example, at least about 0.25 wt %, at least about 0.5 wt %, at least about 1 wt %, at least about 2 wt %, at least about 3 wt %, at least about 5 wt %, at least about 8 wt %, at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the composition can be polyenylphosphatidylcholine. In some cases, the polyenylphosphatidylcholine may be present at a concentration of no more than about 95 wt %, no more than about 90 wt %, no more than about 80 wt %, no more than about 70 wt %, no more than about 65 wt %, no more than about 60 wt %, no more than about 50 wt %, no more than about 40 wt %, no more than about 30 wt %, no more than about 20 wt %, or no more than about 10%. Combinations of any of these are also possible. For instance, the polyenylphosphatidylcholine may be present at between about 8 wt % and about 65 wt %. In some embodiments, at least about 20 wt %, at least about 30 wt %, at least about 40 wt %, at least about 50 wt %, at least about 60 wt %, at least about 70 wt %, at least about 80 wt %, at least about 90 wt %, or about 100 wt % of all of the phosphatidylcholine or lecithin in the composition is polyenylphosphatidylcholine.

While not wishing to be bound to any theory, it is believed that the PPC-enriched phosphatidylcholine may contribute to the stability of the composition, for example, by shielding the tetrahydrobiopterin (or related compound) from oxygen, and/or by enhancing its penetration into the skin or other area, e.g., a mucosal surface.

In certain embodiments, a composition such as those described herein can be formulated to include a first phase and a second phase. Typically, the second phase is substantially immiscible with the first phase comprising phosphatidylcholine or lecithin. Two phases that are substantially immiscible are able to form discrete phases when exposed to each other at ambient conditions (e.g., 25° C. and 1 atm) for extended periods of time (e.g., at least about a day). The phases can be separate identifiable phases (e.g., one may float above the other), or in some cases, the phases are intermingled, e.g., as in an emulsion. The stability of the discrete phases may be kinetic and/or thermodynamic in nature, in various embodiments.

In one set of embodiments, the second phase may comprise an emulsifier which causes the first phase comprising phosphatidylcholine or lecithin to form a liquid crystal, and/or vesicles such as micelles or liposomes. Typically, in a liquid crystal phase, vesicular structures such as micelles, liposomes, hexagonal phases, or lipid bilayers can be formed. In some cases, multilamellar structures may be present within the liquid crystal phase, although in other cases, only unilamellar structures may be present. For example, in certain cases, the PPC-enriched phosphatidylcholine can be loosely arranged in a multilamellar fashion. In some cases, the first phase (e.g., comprising PPC-enriched phosphatidylcholine) and the second phase can form a structure such as is disclosed in U.S. Pat. No. 7,182,956 to Perricone, et al. This is believed (without wishing to be bound by any theory) to form a loosely arranged, yet stable, PPC-enriched phosphatidylcholine-drug complex that may allow penetration and delivery of tetrahydrobiopterin and/or related compounds and optional adjunct ingredients to the skin, e.g., to the dermal vasculature, or to a mucosal surface.

Thus, in some embodiments, the second phase may comprise an emulsifier. The emulsifier, in one embodiment, may be a substance that is able to stabilize an emulsion by increasing its kinetic stability. The emulsifier may also be chosen in some cases to be relatively inert or non-toxic relative to the skin or to a mucosal surface.

A variety of emulsifiers can be used, and many emulsifiers are readily available commercially. In one embodiment, for example, the emulsifier comprises a surfactant. Non-limiting examples of surfactants include a siloxylated polyether comprising dimethyl, methyl(propylpolyethylene oxide propylene oxide, acetate) siloxane commercially available from vendors such as Dow Corning (Dow Corning 190 surfactant). Other examples of materials that can be used as (or within) the second phase (e.g., as emulsifiers) include, but are not limited to, 1,2-propanediol, or silicone fluids containing low viscosity polydimethylsiloxane polymers, methylparaben (p-hydroxy benzoic acid methyl ester) commercially available from vendors such as Dow Corning (Dow Corning 200 silicone fluid). Still other examples include various siloxane or silicone compounds, e.g., hexamethyldisiloxane, amodimethicone, phenyltrimethicone, etc.

In some embodiments, the second phase may comprise a polyglycol. The polyglycol may include a polyhydric alcohol of a monomeric glycol such as polyethylene glycol (PEG) and/or polypropylene glycol (PPG). For example, the PEG or PPG may be PEG or PPG 200, 300, 400, 600, 1,000, 1,450, 3,350, 4,000, 6,000, 8,000, and 20,000, where the number indicates the approximate average molecular weight of the PEG or PPG. As is understood by those of ordinary skill in the art, a polyglycol composition often will comprise a range of molecular weights, although the approximate average molecular weight is used to identify the type polyglycol. More than one PEG and/or PPG can also be present in certain instances.

More than one PEG and/or PPG can also be present in certain instances. The composition can include any suitable amount of polyglycol, for example, at least about 1 wt %, at least about 3 wt %, at least about 5 wt %, at least about 10 wt %, at least about 20 wt %, at least about 30 wt %, at least about 40 wt %, at least about 50 wt %, etc. In some cases, no more than about 60 wt %, no more than about 50 wt %, no more than about 40 wt %, no more than about 30 wt %, no more than about 20 wt %, no more than about 18 wt %, no more than about 15 wt %, no more than about 12 wt %, or no more than about 10 wt % of the composition is polyglycol. Combinations of any of these are also possible. For example, the composition may be between about 0 wt % and about 10 wt % polyglycol. The composition may include one or more than one type of polyglycol.

Additionally, purified water may be added to the second phase in some embodiments, although in other cases, little or no water is present in the second phase. For example, the first phase, the second phase, can contain less than 10%, less than 5%, less than 2%, less than 1%, or less that 0.05% (e.g., wt %) of water relative to the weight of the respective phase or of the entire composition. In some cases, the second phase may also comprise adjunct ingredients such as those described herein.

The second phase may include any one, or more than one, of the materials described above. In addition, any suitable amount of second phase can be used in accordance with various embodiments of the invention. For example, the second phase may be present at at least about 10 wt %, at least about 20 wt %, at least about 30 wt %, at least about 40 wt %, at least about 50 wt %, at least about 60 wt %, at least about 70 wt %, at least about 80 wt %, or at least about 90 wt % of the composition. In some cases, the ratio of the first phase (e.g., comprising phosphatidylcholine or lecithin) to the second phase can be at least about 1:3, at least about 1:2, at least about 1:1, at least about 2:1, at least about 3:1, or at least about 4:1, etc.

As a specific non-limiting example of one set of embodiments, a polyenylphosphatidylcholine comprises a certain material with the trade name NAT 8729, and optionally at least one polyglycol (e.g., PEG or PPG, such as is described herein). The composition can also comprise a PPC-enriched phosphatidylcholine material that is present within the first or second phase, e.g., comprising tetrahydrobiopterin and/or a related compound such as dihydrobiopterin, quinonoid dihydrobiopterin, 7,8-dihydrobiopterin, etc. The second phase may also comprise a surfactant such as a siloxylated polyether comprising dimethyl, methyl(propylpolyethylene oxide propylene oxide, acetate) siloxane commercially available from vendors such as Dow Corning (Dow Corning 190 surfactant) and lubricant such as silicone fluids containing low viscosity polydimethylsiloxane polymers, methylparaben (p-hydroxy benzoic acid methyl ester) commercially available from vendors such as Down Corning (Dow Corning 200 silicone fluid).

Other examples of materials that can be used as (or within) the formulation include, but are not limited to, benzyl alcohol, ethyl alcohol, isopropyl palmitate (IPP), propanediol, and caprylic/capric triglycerides.

As another example, the first phase also comprises, in some embodiments of the invention, a fatty acid ester. Non-limiting examples include ascorbate palmitate or isopropyl palmitate. In some cases, the fatty acid ester is used as a preservative or an antioxidant. The composition can include any suitable amount of fatty acid ester, for example, at least about 1 wt %, at least about 3 wt %, at least about 5 wt %, at least about 10 wt %, at least about 20 wt %, at least about 30 wt %, at least about 40 wt %, at least about 50 wt %, etc. In some cases, no more than about 60 wt %, no more than about 50 wt %, no more than about 40 wt %, no more than about 30 wt %, no more than about 20 wt %, no more than about 18 wt %, no more than about 15 wt %, no more than about 12 wt %, or no more than about 10 wt % of the composition is fatty acid ester. Combinations of any of these are also possible. For example, the composition may be between about 0 wt % and about 10 wt % fatty acid ester. The composition may include one or more than one fatty acid ester.

In another set of embodiments, the composition may also include one or more transdermal penetration enhancers. Examples of transdermal penetration enhancers include, but are not limited to, 1,3-dimethyl-2-imidazolidinone or 1,2-propanediol. Other examples include cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); organic acids (e.g., citric acid); sulfoxides (e.g., dimethylsulfoxide); terpenes (e.g., cyclohexene); ureas; sugars; carbohydrates or other agents. The transdermal penetration enhancers can be present in any suitable amount within the composition. For example, at least about 10 wt %, at least about 20 wt %, at least about 30 wt %, at least about 40 wt %, or at least about 50 wt % of the composition may comprise one or more transdermal penetration enhancers. In some cases, no more than about 60 wt %, no more than about 50 wt %, no more than about 40 wt %, no more than about 30 wt %, no more than about 20 wt %, no more than about 10 wt %, no more than about 9 wt %, or no more than about 5 wt % of the composition comprises transdermal penetration enhancers. Combinations of any of these are also possible. For example, the composition may have between about 0 wt % and about 5 wt % of one or more transdermal penetration enhancers.

In other embodiments, the composition may be modified in order to control depth of penetration. For example, in certain embodiments, the composition includes one or more polymers that act to reduce penetration depth of tetrahydrobiopterin and/or a related compound such as dihydrobiopterin, quinonoid dihydrobiopterin, 7,8-dihydrobiopterin, etc. Controlled depth of penetration may be important for indications where local administration is desired without systemic effects. Examples of transdermal penetration barrier polymers include, but are not limited to, silicone waxes, acrylate polymers, and dimethicone copolymers. In certain embodiments, a transdermal penetration barrier polymer is nonionic. A transdermal penetration barrier polymer can be present in any suitable amount within the composition. For example, at least about 10 wt %, at least about 20 wt %, at least about 30 wt %, at least about 40 wt %, or at least about 50 wt % of the composition may comprise one or more transdermal penetration barrier polymers. In some cases, no more than about 60 wt %, no more than about 50 wt %, no more than about 40 wt %, no more than about 30 wt %, no more than about 20 wt %, no more than about 10 wt %, no more than about 9 wt %, or no more than about 5 wt % of the composition comprises a transdermal penetration barrier polymer. Combinations of any of these are also possible. For example, the composition may have between about 0 wt % and about 5 wt % of one or more transdermal penetration barrier polymers.

In some embodiments, various compositions of the invention are formulated to be substantially clear or substantially transparent. Transparency may be useful, for instance, for product acceptance in the marketplace, e.g., when applied to the skin of a subject. However, in other embodiments, the composition is not necessarily transparent. Certain substances can be useful in providing a substantially transparent composition, for example, fatty acid esters such as ascorbate palmitate or isopropyl palmitate. In one set of embodiments, the composition may be substantially transparent such that incident visible light (e.g., have wavelengths of between about 400 nm and about 700 nm) can be transmitted through 1 cm of the composition with a loss in intensity of no more than about 50%, about 60%, about 70%, about 80%, or about 90% relative to the incident light. In some embodiments, there may be no substantial difference in the wavelengths that are absorbed by the composition (i.e., white light passing through the composition appears white), although in other cases, there can be more absorption at various wavelengths (for example, such that white light passing through the composition may appear colored).

Other components may also be present within the composition, in accordance with certain embodiments of the invention. For example, the composition may include volatile organic fluids, fatty acids, volatile aromatic cyclic compounds, high molecular weight hydrocarbons, or the like.

As mentioned above, some aspects of the invention include nitric oxide (NO) in addition to tetrahydrobiopterin and/or a related compound. (However, in other aspects, it should be understood that the composition may be free of any nitric oxide.) Without wishing to be bound by theory, it is believed that nitric oxide forms reversible physical bonds, similar to hydrogen bonds or van der Waals forces, with phosphatidylcholine or other lecithin molecules, e.g., containing one or more double bonds, which may allow nitric oxide to become entrapped and thereby remain intact for an extended period of time, e.g., during storage. These physical bonds, however, are believed to be not very stable, and may in some cases be easily broken up, for example, upon various physical agitations such as rubbing the composition against skin or a mucosal surface, thereby releasing the entrapped nitric oxide. While others have stabilized other substances or drugs within phosphatidylcholine or lecithin compositions or vesicles, for example, protein drugs such as insulin, it is surprising that a small, highly reactive molecule such as NO could similarly be stabilized, especially when it would have been expected that a molecule as small as NO would readily diffuse away from such compositions and/or would have reacted with water that is typically present within such compositions.

In some embodiments, nitric oxide not only can be entrapped in phosphatidylcholine or lecithin compositions such as those described herein, but also that such entrapped compositions may have a long shelf life, especially when refrigerated. No or little loss or reaction of nitric oxide is expected during extended refrigerated storage, at least under certain conditions.

In some embodiments, the tetrahydrobiopterin and/or related compounds within the composition may be stable at room temperature, e.g., preventing or reducing exposure of the tetrahydrobiopterin (or related compound) to oxygen, e.g., within the air. In some cases, the tetrahydrobiopterin may be released, for example, when the composition is exposed to an aqueous environment, e.g., within the body. Without wishing to be bound by any theory, it is believed that when the composition is applied to the skin, the liquid crystal structure collapses, delivering tetrahydrobiopterin and/or related compounds to, e.g., a muscle or other desired area of treatment. The concentration of tetrahydrobiopterin and/or related compounds inside the liquid crystal matrix can be varied in terms of concentration. The matrix also may act as a sustained release delivery system in some embodiments. It is also believed that the liquid crystal is highly penetrating, such that tetrahydrobiopterin and/or related compounds can be delivered to the epidermis, dermis and dermal vascular for systemic release as well as to subcutaneous fat, at least under some conditions.

Thus, a composition such as is discussed herein may be prepared and/or stored at any suitable temperature and under any suitable conditions. In some embodiments, for instance, a composition can be prepared and/or stored under limited or no oxygen conditions, as oxygen can adversely react with tetrahydrobiopterin (or related compound). The composition can also be prepared and/or stored under limited or no nitrogen and/or carbon dioxide. For instance, the composition may be prepared and/or stored in a sealed environment (e.g., stored in a sealed container). The sealed environment (e.g., container) can be at least substantially devoid of gas, and/or contains a gaseous mixture that excludes, or at least is depleted in, oxygen. In some embodiments, an environment depleted in oxygen may have less than about 20%, less than about 15%, less than about 10%, less than about 5%, about 1% or less, about 0.1% or less, about 0.01% or less, about 0.001% or less, oxygen (e.g., as a wt % or as molar % per volume). For example, the gaseous mixture may include a noble gas, such as argon, helium, neon, etc. In one set of embodiments, the container may comprise a multi-layered metallic and/or polymeric barrier, e.g., formed from Glaminate® (American Can Company). For instance, the container may have the shape of a tube. Thus, in certain embodiments, the container is substantially resistant to oxygen permeation, nitrogen permeation, and/or carbon dioxide permeation. In certain embodiments, the container is substantially watertight, for example, such that substantially no water is absorbed by the container, or such that no water is able to pass through the container even if the container is filled with water.

In certain embodiments, the composition may be stored at temperatures of less than about 80° C., less than about 70° C., less than about 60° C., less than about 50° C., less than about 40° C., less than about 30° C., less than about 25° C., less than about 20° C., less than about 15° C., less than about 10° C., less than about 5° C., less than about 0° C., etc., for extended periods of time, e.g., at least about a day, at least about a week, at least about 4 weeks, at least about 6 months, etc.

In accordance with certain aspects of the invention, a composition as discussed herein may be prepared by mixing a first phase and a second phase together. The second phase can comprise an emulsifier, or any other components discussed herein. The first phase may comprise a lecithin such as phosphatidylcholine and/or polyenylphosphatidylcholine, e.g., PPC-enriched phosphatidylcholine, for instance, as described herein. In some embodiments, other components are also mixed into the composition, for example, transdermal penetration enhancers, adjuvants, polyglycols (e.g., PEG and/or PPG), surfactants, lubricants, etc. as discussed herein.

In some embodiments, tetrahydrobiopterin and/or a related compound such as dihydrobiopterin, quinonoid dihydrobiopterin, 7,8-dihydrobiopterin, etc. may be added to a composition during or after the formulation of any phase or composition as described herein, e.g., by routine methods known in the art. For example, tetrahydrobiopterin (or related compound) may be added to any phase of a formulation or composition, or after any formulation or composition described herein is made.

In addition, in some (but not all) embodiments, the composition may include nitric oxide as well. The nitric oxide may be added, for example, by passing bubbles of nitric oxide through the composition. See, for example, U.S. Pat. No. 8,668,937, incorporated herein by reference in its entirety.

In one aspect, application of tetrahydrobiopterin and/or a related compound such as dihydrobiopterin, quinonoid dihydrobiopterin, 7,8-dihydrobiopterin, etc., e.g., in a composition as described herein, may be applied to the skin of a subject, e.g., one having or at risk of a disease, condition, or need described herein. Additionally, in some embodiments, the composition may be applied in conjunction with other types of treatments to a subject, e.g., to the skin of a subject, for treatment of any of the diseases, conditions, or needs described herein. These may be occur, e.g., simultaneously or sequentially, in various embodiments. Thus, certain compositions as described herein may be used to treat a wide variety of diseases or conditions. To “treat” a disorder means to reduce or eliminate a sign or symptom of the disorder, to stabilize the disorder, and/or to reduce or slow further progression of the disorder. The subject may be a human subject, or a non-human mammal in some cases.

For example, in one set of embodiments, a composition such as is described herein may be applied to a subject to promote hair growth, for example in a region where hair growth is desired. In some embodiments, a composition can be applied to the skin of a subject, for example, to prevent or reduce hair loss, for example in a region where hair loss is undesirable. In some embodiments, a region targeted for hair growth and or a reduction in hair loss may be the skin surface of the scalp. In some embodiments, a subject may be a human. The subject may be male or female. In some embodiments, a subject may have lost hair (e.g., is bald, balding, or has thinning hair) and/or may be at risk of losing hair (e.g., due to age, a genetic risk and/or family history of hair loss). In some embodiments, the subject may have lost hair or be at risk for losing hair due to injury (e.g., trauma or burn) or due to a disease and/or treatment (e.g., chemotherapy).

In another set of embodiments, a composition such, as described herein can be applied to the skin of a subject, for example, to a wound to promote wound healing. In some embodiments, certain compositions of the invention can be used to promote any wound healing by applying the composition to an existing wound. It should be appreciated that certain compositions of the invention may be used to treat or promote healing of any type of wound, including cuts, scrapes, other traumatic wounds, burns or other accidental wounds. Non-limiting examples of wounds include wound can be an anal fissure, a surgical site, a trauma site, a burn, an abrasion, a sunburn, a cut or laceration on the skin, or any other damaged region of the skin. In some embodiments, the composition is applied to a mucosal surface of the subject, for example, to the nose. Accordingly, certain compositions of the invention also may be used to treat wounds that result from surgical intervention (e.g., any medical intervention or operation that requires wound healing as part of the recovery process). In some cases, certain compositions of the invention can be used prophylactically to a site (for example a surgical site) in anticipation of a wound. In some embodiments, a composition such as described herein can be used to prepare a tissue site for wound healing prior to the wound (e.g., prior to surgery) and/or after the wound (e.g., and/or after surgery). In some embodiments, a composition such as is described herein may be applied to the surface of a wound or to skin prior to a wound.

Yet another set of embodiments is generally directed to treating erectile dysfunction or sexual dysfunction. For example, a composition such as described herein can be applied to the skin of a subject on or near a male or female genital region to treat sexual dysfunction and/or to enhance sexual performance or experience. In some embodiments, a composition is applied to the perineal region (e.g., penis), to the vulva, etc., to release tetrahydrobiopterin (or related compound) to the skin and underlying tissue directly upon topical administration of the composition to the subject. For example, a composition of the invention may be applied to the genital perineal region or penis or portion thereof of a male subject to treat erectile dysfunction, to the vulva of a female subject to treat sexual dysfunction (or to any other portion at or near the genital perineal region in male or female subjects).

In still another set of embodiments, a composition as described herein is used to treat acne or other conditions. Thus, in one set of embodiments, the application of a composition as is described herein to acne lesions and the surrounding skin may result in clinical improvement. Without wishing to be bound by any theory, it is believed that nitric oxide (e.g., stimulated upon application of tetrahydrobiopterin and/or a related compound) may block cytokine production, and therefore the primary event of interleukin-causing retention hyperkeratosis may be avoided. In addition, in some cases, the natural antioxidant and anti-inflammatory effects of nitric oxide may prevent the magnifying cascade that results in clinically apparent inflammatory lesions. Also, anti-microbial effects of nitric oxide may be used to clear follicles of secondary infection, e.g., from propionibacteria.

In another set of embodiments, a composition as described herein may be applied topically to an infected area of skin or other soft tissue, e.g. to treat an infection. The composition may be applied in some cases without the necessity of moisture or dressings. Without wishing to be bound by any theory, it is believed that nitric oxide (e.g., stimulated upon application of tetrahydrobiopterin and/or a related compound) is an effective antimicrobial agent of Staphylococcus aureus, and/or other infectious organisms.

According to yet another set of embodiments, a variety of inflammatory diseases, such as inflammatory dermatoses may be treated with a composition as described herein, e.g., containing tetrahydrobiopterin and/or a related compound. Without wishing to be bound by any theory, it is believed that increased levels of nitric oxide (e.g., stimulated upon application of tetrahydrobiopterin and/or a related compound) can mediate or attenuate the inflammatory component of various inflammatory diseases, such as inflammatory dermatoses, osteoarthritis, and septic shock. Inflammatory dermatoses are generally characterized by inflammation in the epidermis and/or dermis. Examples of inflammatory dermatoses include, but are not limited to, psoriasis, atopic dermatitis, eczematous dermatitis, dermatitis, eczema, contact dermatitis, erythema multiforme, pruritus (urticaria), and the like. Some inflammatory dermatoses are autoimmune in nature. In many cases, lesions or eruptions may appear on the skin, and may be acute (lasting days to weeks) or chronic (lasting months to years). Acute lesions are relatively common and exhibit a wide range of clinical conditions. Usually, these conditions are triggered by local or systemic immunologic factors (e.g., allergic reaction).

According to still another set of embodiments, the application of a composition as described herein may be used to treat cardiovascular conditions such as arteriosclerosis, hypertension, sickle-cell anemia, etc. Without wishing to be bound by any theory, it is believed that increased levels of nitric oxide (e.g., stimulated upon application of tetrahydrobiopterin and/or a related compound) may be used to treat the symptoms of a cardiovascular disorder, such as arteriosclerosis, hypertension, sickle-cell anemia, etc. The nitric oxide may act as an antioxidant, an anti-inflammatory, and/or an anti-proliferative, e.g., at needed physiologic levels. In some cases, nitric oxide may be used to cause arterial relaxation, e.g., for treating hypertension or sickle-cell anemia. Endothelial dysfunction in coronary arteries leads to many of the acute coronary syndromes such as unstable angina and myocardial infarction. Thus, increased levels of nitric oxide would have therapeutic effects within coronary arteries, which may be used to treat or prevent vasospastic and occlusive events.

In yet another set of embodiments, a composition as described herein is applied topically to the skin, e.g., to treat or prevent a disease or condition characterized by a learning or memory disorder, and/or to enhance learning or memory. For example, the composition may be applied to the carotid arteries, e.g., for delivery into the head or the brain. Examples of learning and memory disorders include, but are not limited to, agnosia, Alzheimer's disease, amnesia, mild cognitive impairment (MCI), traumatic brain injury, dementia, Huntington's Disease, Parkinson's Disease, or Wernicke-Korsakoff's Syndrome. In addition, in some cases, the condition may be mentally retardation. In yet another set of embodiments, the subject may be normal (or not indicated as having a learning or memory disorder), but wish to enhance his or her learning or memory abilities. Without wishing to be bound by any theory, it is believed that nitric oxide (e.g., stimulated upon application of tetrahydrobiopterin and/or a related compound) generally acts as a neurotransmitter in the brain, and thus, the application of compositions such as those described herein may enhance neuronal function within the brain, thereby enhancing learning or memory, which can be used to facilitate treatment of learning or memory disorders.

According to another set of embodiments, a composition as described herein is used to treat muscular and/or neuromuscular diseases or other conditions. Without wishing to be bound by any theory, it is believed that nitric oxide (e.g., stimulated upon application of tetrahydrobiopterin and/or a related compound) may increase vasodilation, allowing for muscle growth and/or repair.

In another set of embodiments, a composition as described herein is used to treat osteoporosis or other conditions. Without wishing to be bound by any theory, it is believed that nitric oxide (e.g., stimulated upon application of tetrahydrobiopterin and/or a related compound) is beneficial in the treatment of osteoporosis due to the relationship between the NO-cGMP pathway and bone health. Supplementation with nitric oxide in deficient individuals may allow for biological homeostasis.

In accordance with still another set of embodiments, a composition as described herein is used to treat neuropathic pain or other conditions. Without wishing to be bound by any theory, it is believed that impaired or reduced nitric oxide is involved in the pathogenesis of neuropathic pain, e.g., diabetic neuropathic pain. Also, the vasodilation effects of nitric oxide may induce angiogenesis of the vasa nervorum, increasing the analgesic effect of nitric oxide on neuropathic pain, in some embodiments.

In yet another set of embodiments, a composition as described herein is used to treat cancer or other conditions. In some embodiments, a composition described herein is administered as an adjuvant to traditional chemotherapy. Without wishing to be bound by any theory, it is believed that nitric oxide (e.g., stimulated upon application of tetrahydrobiopterin and/or a related compound) is effective in treating cancer, such as prostate cancer, as evidenced by studies with nitric oxide donors such as nitroglycerin. In addition, nitric oxide can enhance cytotoxic efficacy of some chemotherapeutic agents and radiation. Exemplary cancers include non-solid tumors such as leukemia, for example acute myeloid leukaemia, multiple myeloma, haematologic malignancies or lymphoma, and also solid tumors and their metastases such as melanoma, non-small cell lung cancer, glioma, hepatocellular (liver) carcinoma, glioblastoma, carcinoma of the thyroid, cholangiocarcinoma, bile duct, bone, gastric, brain/CNS, head and neck, hepatic, stomach, prostate, breast, renal, testicular, ovarian, cervix, skin, cervical, lung, muscle, neuronal, oesophageal, bladder, lung, uterine, vulval, endometrial, kidney, colon, colorectal, pancreatic, pleural/peritoneal membranes, salivary gland, epidermoid tumors and hematological malignancies. In certain embodiments, the cancer is prostate cancer. In certain embodiments, the cancer is skin cancer (e.g., basal cell carcinoma, squamous cell carcinoma, melanoma).

In addition, various non-limiting examples of systems and methods of treatment of a disease or condition using a formulation comprising nitric oxide are disclosed in the following U.S. patent applications, each filed Mar. 13, 2013, and, each incorporated by reference in its entirety: “Systems and Methods for Delivery of Peptides” (U.S. patent application Ser. No. 13/801,402) “Treatment of Skin, Including Aging Skin, to Improve Appearance” (U.S. patent application Ser. No. 13/801,446); “Hair Treatment Systems and Methods Using Peptides and Other Compositions” (U.S. patent application Ser. No. 13/801,488); “Skin Tanning Using Peptides and Other Compositions” (U.S. patent application Ser. No. 13/801,518); “Topical Systems and Methods for Treating Sexual Dysfunction” (U.S. patent application Ser. No. 13/801,543); “Immune Modulation Using Peptides and Other Compositions” (U.S. patent application Ser. No. 13/800,952); “Cardiovascular Disease Treatment and Prevention” (U.S. patent application Ser. No. 13/801,013); “Wound Healing Using Topical Systems and Methods” (U.S. patent application Ser. No. 13/801,061); “Peptide Systems and Methods for Metabolic Conditions” (U.S. patent application Ser. No. 13/801,110); “Methods and Systems for Treating or Preventing Cancer” (U.S. patent application Ser. No. 13/801,188); “Compositions and Methods for Affecting Mood States” (U.S. patent application Ser. No. 13/081,240); “Improvement of Memory or Learning Using Peptide and Other Compositions” (U.S. patent application Ser. No. 13/801,298); and “Brain and Neural Treatments Comprising Peptides and Other Compositions” (U.S. patent application Ser. No. 13/801,345).

In certain aspects of the invention, a composition such as those described herein can be administered to a subject, such as a human subject, by rubbing it on the skin or a mucosal surface of the subject, e.g., in areas located at or at least within the vicinity of a desired target area. Other areas have also been described herein, in other embodiments. Without wishing to be bound by any theory, it is believed that phosphatidylcholine provides or facilitates delivery of tetrahydrobiopterin and/or a related compound such as dihydrobiopterin, quinonoid dihydrobiopterin, 7,8-dihydrobiopterin, etc. to the skin or a mucosal surface, and/or to tissues below the skin or mucosal surface, allowing the tetrahydrobiopterin and/or related compounds to be delivered to a target area. In some embodiments, the composition can be applied, by rubbing the composition topically against the skin, or to the mucosal surface, which allows the composition (or at least, tetrahydrobiopterin and/or related compounds) to be absorbed by the skin or mucosal surface. The composition can be applied once, or more than once. For example, the composition may be administered at predetermined intervals. In some embodiments, for instance, the composition may be applied once per day, twice per day, 3 times per day, 4 times per day, once every other day, once every three days, once every four days, etc. The amount of tetrahydrobiopterin and/or related compounds necessary to bring about the therapeutic treatment is not fixed per se, and may depend upon factors such as the desired outcome, the type and severity the disease or condition, the form of tetrahydrobiopterin, the concentration of tetrahydrobiopterin and/or related compounds present within the composition, etc.

Thus, some embodiments of the invention provide methods of administering any composition such as discussed herein to a subject. When administered, the compositions of the invention are applied in a therapeutically effective, pharmaceutically acceptable amount as a pharmaceutically acceptable formulation. Any of the compositions of the present invention may be administered to the subject in a therapeutically effective dose. When administered to a subject, effective amounts will depend on the particular condition being treated and the desired outcome. A therapeutically effective dose may be determined by those of ordinary skill in the art, for instance, employing factors such as those described herein and using no more than routine experimentation.

In certain embodiments of the invention, the administration of various compositions of the invention may be designed so as to result in sequential exposures to the composition over a certain time period, for example, hours, days, weeks, months, or years. This may be accomplished, for example, by repeated administrations of a composition of the invention by one or more of the methods described herein, or by a sustained or controlled release delivery system in which the composition is delivered over a prolonged period without repeated administrations. Administration of the composition using such a delivery system may be, for example, by a transdermal patch. Maintaining a substantially constant concentration of the composition may be preferred in some cases.

For certain chronic treatments or therapies, it is contemplated that a composition as discussed herein may be used to deliver tetrahydrobiopterin and/or a related compound such as dihydrobiopterin, quinonoid dihydrobiopterin, 7,8-dihydrobiopterin, etc. to the skin or mucosal surface at a relatively high concentration during an initial treatment, and the amount of tetrahydrobiopterin and/or related compounds may be lowered or “titrated” down to a relatively lower concentration maintenance dose or amount. A composition comprising tetrahydrobiopterin and/or related compounds as described herein can be used to promote vasodilation of blood vessels within and/or under the skin. As a specific example, a composition described herein may be used for certain treatments of hair loss or wounds, e.g., anal fissures. In some cases, gradual improvement may be observed with successive applications.

In some embodiments, an effective amount is an amount sufficient to have a measurable positive effect on blood flow and/or vasodilation, and/or a measurable negative effect on blood pressure. In some embodiments, the effect on blood flow and/or vasodilation is observed local to the site of topical application. In some embodiments, an effective amount is an amount sufficient to have a measurable effect on immune modulation such as is described herein.

In one set of embodiments, compositions described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

In one set of embodiments, the dosage may be between about 0.01 mg and about 500 g, between about 0.01 mg and about 300 g, between about 0.01 mg and about 100 g, between about 0.01 mg and about 30 g, between about 0.01 mg and about 10 g, between about 0.01 mg and about 3 g, between about 0.01 mg and about 1 g, between about 0.01 mg and about 300 mg, between about 0.01 mg and about 100 mg, between about 0.01 mg and about 30 mg, between about 0.01 mg and about 10 mg, between about 0.01 mg and about 3 mg, between about 0.01 mg and about 1 mg, between about 0.01 mg and about 0.3 mg, or between about 0.01 mg and about 0.1 mg.

In another set of embodiments, the dosage may be at least about 0.01 mg, at least about 0.02 mg, at least about 0.03 mg, at least about mg, at least about 0.05 mg, at least about 0.1 mg, at least about 0.2 mg, at least about 0.3 mg, at least about 0.5 mg, at least about 1 mg, at least about 2 mg, at least about 3 mg, at least about 5 mg, at least about 10 mg, at least about 20 mg, at least about 30 mg, at least about 50 mg, at least about 100 mg, at least about 200 mg, at least about 300 mg, at least about 500 mg, at least about 1 g, at least about 2 g, at least about 3 g, at least about 5 g, at least about 10 g, etc. In some cases, the dosage may be no more than about 10 g, no more than about 5 g, no more than about 3 g, no more than about 2 g, no more than about 1 g, no more than about 500 mg, no more than about 300 mg, no more than about 200 mg, no more than about 100 mg, no more than about 50 mg, no more than about 30 mg, no more than about 20 mg, no more than about 10 mg, no more than about 5 mg, no more than about 3 mg, no more than about 2 mg, no more than about 1 mg, no more than about 0.5 mg, no more than about 0.3 mg, no more than about 0.2 mg, no more than about 0.1 mg, no more than about 0.05 mg, no more than about 0.03 mg, no more than about 0.02 mg, no more than about 0.01 mg, etc. In some cases, combinations of any of these are also possible, e.g., between about 0.01 mg and about 0.1 mg.

The compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent. In certain embodiments, the additional therapeutic agent is present in a provided composition in addition to tetrahydrobiopterin and/or a related compound such as dihydrobiopterin, quinonoid dihydrobiopterin, 7,8-dihydrobiopterin, etc. In other embodiments, the additional therapeutic agent is administered separately from the composition comprising tetrahydrobiopterin and/or related compounds.

When co-administered with other agents, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed.

In certain embodiments, a composition comprising tetrahydrobiopterin and/or a related compound such as dihydrobiopterin, quinonoid dihydrobiopterin, 7,8-dihydrobiopterin, etc. as described herein, and the additional therapeutic agent are each administered in an effective amount (i.e., each in an amount which would be therapeutically effective if administered alone). In other embodiments, a composition comprising tetrahydrobiopterin and/or related compounds as described herein, and the additional therapeutic agent are each administered in an amount which alone does not provide a therapeutic effect (a sub-therapeutic dose). In yet other embodiments, a composition comprising tetrahydrobiopterin (or a related compound) as described herein can be administered in an effective amount, while the additional therapeutic agent is administered in a sub-therapeutic dose. In still other embodiments, a composition comprising tetrahydrobiopterin (or a related compound) as described herein can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order. When co-administration involves the separate administration of the first amount of a composition as described herein, and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound. For example, a composition as described herein, and the second therapeutic agent can be administered in any order within about 24 hours of each other, within about 16 hours of each other, within about 8 hours of each other, within about 4 hours of each other, within about 1 hour of each other or within about 30 minutes of each other.

More specifically, a first therapy (e.g., a prophylactic or therapeutic agent such as a composition described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In one set of embodiments, a composition such as is discussed herein may be applied to the skin or mucosal surface of a subject, e.g., at any suitable location. The composition may be contacted using any suitable method. For example, the composition may be rubbed on, poured on, applied with an applicator (e.g., a gauze pad, a swab, a bandage, etc.), or the like. In some cases, the composition can be a liquid, a gel, a cream, a lotion, an ointment, a solid “stick,” or the like, that can be applied to the skin or mucosal surface by hand, for example, by rubbing or spraying. In addition, in certain embodiments, the composition is applied to a mucosal surface of the subject. For example, the composition may be applied to the nose or nostrils, the mouth, the lips, the eyelids, the ears, the genital area (of either male or female subjects), or the anus.

The composition may be applied, in certain embodiments, at or near a genital region of the skin of a male or a female subject, e.g., to treat sexual dysfunction. For example, the composition may be applied to the penis of a male subject or to the vulva of a female subject, or to any other suitable genital perianal region. In certain embodiments, for instance, the composition is used to treat erectile dysfunction. In some cases, the composition can be applied to a condom or other suitable sexual aid. The composition can be applied, for example, just before the subject engages in sexual activity.

The compositions of the present invention may additionally comprise one or more adjunct ingredients, for instance, pharmaceutical drugs or skin care agents. For example, compositions of the invention may include adjuvants such as salts, buffering agents, diluents, excipients, chelating agents, fillers, drying agents, antioxidants, antimicrobials, preservatives, binding agents, bulking agents, silicas, solubilizers, or stabilizers. Non-limiting examples include species such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable concentrations can be determined by those of ordinary skill in the art, using no more than routine experimentation. Those of ordinary skill in the art will know of other suitable formulation ingredients, or will be able to ascertain such, using only routine experimentation.

Preparations can include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, organic esters such as ethyl oleate, or fixed oils including synthetic mono or di-glycerides. Aqueous solvents include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, a composition such as described herein may be applied to a surgical device, tool, or other substrate. For example, a composition of the invention may be applied to sutures, implants, surgical tools, or other substrates that may come into contact with wounded tissue (e.g., cut tissue) during surgery. In some embodiments, a composition may be provided as a cream or ointment as described in more detail herein. It also should be appreciated that certain compositions of the invention may be provided on surgical dressings, bandages, or other material that is to be contacted to a surgical wound.

In one set of embodiments, a composition such as is described herein may be applied to a material or substrate immediately prior to use on a subject. However, in some embodiments, a material or substrate may be prepared (e.g., packaged, stored, or otherwise prepared) to contain a composition prior to use. For example, prepackaged bandages or surgical devices, sutures, or implants may be prepared and packaged with a coating of a composition such as is described herein. Compositions of the invention may be used for human or other animal subjects (male or female).

In another aspect, the present invention is directed to a kit including one or more of the compositions discussed herein. A “kit,” as used herein, typically defines a package or an assembly including one or more of the compositions of the invention, and/or other compositions associated with the invention, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the composition and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The following documents are incorporated herein by reference: U.S. Pat. No. 8,668,937, issued Mar. 11, 2014, entitled “Topical Nitric Oxide Systems and Methods of Use Thereof”; U.S. Pat. No. 8,435,942, issued Apr. 26, 2006, entitled “Methods for Formulating Stabilized Insulin Compositions”; U.S. Pat. No. 7,182,956, issued Feb. 27, 2007, entitled “Stable Topical Drug Delivery Compositions”; U.S. Pat. No. 8,273,711, issued Sep. 25, 2012, entitled “Topical Drug Delivery Using Phosphatidylcholine”; U.S. patent application Ser. No. 13/801,402, filed Mar. 13, 2013, entitled “Systems and Methods for Delivery of Peptides”; U.S. patent application Ser. No. 13/801,446, filed Mar. 13, 2013, entitled “Treatment of Skin, Including Aging Skin, to Improve Appearance”; U.S. patent application Ser. No. 13/801,488, filed Mar. 13, 2013, entitled “Hair Treatment Systems and Methods Using Peptides and Other Compositions”; U.S. patent application Ser. No. 13/801,518, filed Mar. 13, 2013, entitled “Skin Tanning Using Peptides and Other Compositions”; U.S. patent application Ser. No. 13/801,543, filed Mar. 13, 2013, entitled “Topical Systems and Methods for Treating Sexual Dysfunction”; U.S. patent application Ser. No. 13/800,952, filed Mar. 13, 2013, entitled “Immune Modulation Using Peptides and Other Compositions”; U.S. patent application Ser. No. 13/801,013, filed Mar. 13, 2013, entitled “Cardiovascular Disease Treatment and Prevention”; U.S. patent application Ser. No. 13/801,061, filed Mar. 13, 2013, entitled “Wound Healing Using Topical Systems and Methods”; U.S. patent application Ser. No. 13/801,110, filed Mar. 13, 2013, entitled “Peptide Systems and Methods for Metabolic Conditions”; U.S. patent application Ser. No. 13/801,188, filed Mar. 13, 2013, entitled “Methods and Systems for Treating or Preventing Cancer”; U.S. patent application Ser. No. 13/801,240, filed Mar. 13, 2013, entitled “Compositions and Methods for Affecting Mood States”; U.S. patent application Ser. No. 13/801,298, filed Mar. 13, 2013, entitled “Improvement of Memory or Learning Using Peptide and Other Compositions”; U.S. patent application Ser. No. 13/801,345, filed Mar. 13, 2013, entitled “Brain and Neural Treatments Comprising Peptides and Other Compositions”; U.S. patent application Ser. No. 13/019,101, filed Feb. 1, 2011, entitled “Method of Delivering Stable Topical Drug Compositions”; U.S. patent application Ser. No. 13/926,688, filed Jun. 25, 2013, entitled “Topical Drug Delivery Using Phosphatidylcholine”; Int. Pat. Apl. Ser. No. PCT/US2014/025822, filed Mar. 13, 2014, entitled “Treatment of Skin, Including Aging Skin, to Improve Appearance”; Int. Pat. Apl. Ser. No. PCT/US2014/025913, filed Mar. 13, 2014, entitled “Immune Modulation Using Peptides and Other Compositions”; Int. Pat. Apl. Ser. No. PCT/US2014/025996, filed Mar. 13, 2014, entitled “Cardiovascular Disease Treatment and Prevention”; Int. Pat. Apl. Ser. No. PCT/US2014/025572, filed Mar. 13, 2014, entitled “Wound Healing Using Topical Systems and Methods”; Int. Pat. Apl. Ser. No. PCT/US2014/025630, filed Mar. 13, 2014, entitled “Peptide Systems and Methods for Metabolic Conditions”; Int. Pat. Apl. Ser. No. PCT/US2014/025758, filed Mar. 13, 2014, entitled “Methods and Systems for Treating or Preventing Cancer”; Int. Pat. Apl. Ser. No. PCT/US2014/025898, filed Mar. 13, 2014, entitled “Improvement of Memory or Learning Using Peptide and Other Compositions”; Int. Pat. Apl. Ser. No. PCT/US2014/025820, filed Mar. 13, 2014, entitled “Brain and Neural Treatments Comprising Peptides and Other Compositions”; and Int. Pat. Apl. Ser. No. PCT/US2014/025705, filed Mar. 13, 2014, entitled “Systems and Methods for Delivery of Peptides.”

The following are also incorporated herein by reference in their entireties: U.S. patent application Ser. No. 14/492,227, filed Sep. 22, 2014, entitled “Prevention and Treatment of Cardiovascular Diseases Using Systems and Methods for Transdermal Nitric Oxide Delivery”; U.S. patent application Ser. No. 13/801,231, filed Mar. 13, 2013, entitled “Prevention and Treatment of Cardiovascular Diseases Using Systems and Methods for Transdermal Nitric Oxide Delivery”; U.S. patent application Ser. No. 13/623,018, filed Sep. 19, 2012, entitled “Prevention and Treatment of Cardiovascular Diseases Using Systems and Methods for Transdermal Nitric Oxide Delivery”; and U.S. Provisional Patent Application Ser. No. 62/081,175, filed Nov. 18, 2014, entitled “Systems and Methods for Delivery of Tetrahydrobiopterin and Related Compounds.”

The following examples are intended to illustrate certain embodiments of the present invention, but do not exemplify the full scope of the invention.

### Example 1

This example illustrates techniques for preparing compositions in accordance with one or more embodiments of the invention. Specifically, four formulations suitable as a tetrahydrobiopterin carrier are provided: two four-phase formulations (HNC 156-43, HNC 156-50), and two single-phase formulations (HNC 156-47, HNC 159-136).

### HNC 156-43

This formulation is formed of: 77.7% water, 6.0% Phospholipon-90G (American Lecithin Company), 0.10% EDTA-Na₂ (Sigma), 0.1% citric acid, 5.0% isopropyl palmitate (IPP, Kraft Chemicals), 5.0% Promulgen-D (Lubrizol), 3.0% Arlacel-165 (Croda), 1.0% cetearyl alchohol 50/50, 0.5% Dow Corning Fluid 200-10 CST, 0.1% Tocotrienol-50C (Carotech), 0.5% Optiphen Plus (Lotioncrafter), and 1.0% Seppitonic M3 (Seppic).

The individual ingredients are divided between four phases as follows:

Phase 1: water, Phospholipon-90G, EDTA-Na₂, citric acid.

Phase 2: IPP, Promulgen-D, Arlacel-165, cetearyl alchohol 50/50, Dow Corning Fluid 200-10 CST, Tocotrienol-50C.

Phase 3: Optiphen Plus.

Phase 4: Seppitonic M3.

### HNC 156-50

This formulation is formed of: 78.3% water, 10.0% Phospholipon-90G (American Lecithin Company), 0.10% EDTA-Na₂ (Sigma), 0.2% sodium hyaluronate (1% solution), 3.0% isopropyl palmitate (IPP, Kraft Chemicals), 3.0% Promulgen-D (Lubrizol), 4.0% Arlacel-165 (Croda), 0.7% cetearyl alchohol 50/50, 0.5% Optiphen Plus (Lotioncrafter), and 0.2% dimethylethanolamine (DMAE, Sigma).

The individual ingredients are divided between four phases as follows:

Phase 1: water, Phospholipon-90G, EDTA-Na₂, sodium hyaluronate.

Phase 2: IPP, Promulgen-D, Arlacel-165, cetearyl alchohol 50/50.

Phase 3: Optiphen Plus.

Phase 4: DMAE.

The four-phase compositions are generally prepared as follows: Phases 1 and 2 are heated to 60° C., added and mixed together, then allowed to cool to 48° C., at which point Phase 3 is added and mixed. The mixture was then allowed to cool to 38° C., at which point phase 4 is added and mixed.

### HNC 156-47

This formulation is formed of: 47% water, 5.0% PEG-200 (Sigma), 45% Phospholipon-90G (American Lecithin Company), 1.0% benzyl alcohol, and 2.0% ethyl alcohol.

### HNC 159-136

This formulation is formed of: 65% Phospholipon-90G (American Lecithin Company), 18.0% isopropyl palmitate (IPP, Kraft Chemicals), 8% capric caprylic triglycerides (RITA Corp.), and 9% propanediol (Dupont).

Any of the above compositions are effective as carriers for tetrahydrobiopterin. For example, the tetrahydrobiopterin may be incorporated into each of the above formulations at a concentration of 5.0 mg/cc.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article for transdermal delivery, the article comprising:
a composition comprising a carrier and lecithin, the lecithin containing tetrahydrobiopterin and/or a salt thereof, wherein the composition further comprises no more than about 250 ppm of water by weight of the composition.

2. The article of claim 1, wherein the composition comprises tetrahydrobiopterin.

3. The article of claim 1, wherein the composition comprises a tetrahydrobiopterin salt.

4. (canceled)

5. The article of claim 1, wherein the tetrahydrobiopterin and/or salt thereof is present at at least about 0.5% by weight.

6-7. (canceled)

8. The article of claim 1, wherein the composition further comprises molecular nitric oxide.

9-10. (canceled)

11. The article of claim 1, wherein the lecithin is present at at least about 0.25% by weight.

12-16. (canceled)

17. The article of claim 1, wherein the lecithin comprises a phosphatidylcholine.

18-20. (canceled)

21. The article of claim 17, wherein at least some of the phosphatidylcholine comprises a polyenylphosphatidylcholine.

22-24. (canceled)

25. The article of claim 1, wherein the lecithin is present as a liquid crystal structure.

26-35. (canceled)

36. The article of claim 1, wherein the composition is a gel.

37-40. (canceled)

41. An article for transdermal delivery, the article comprising:
a composition comprising a carrier and lecithin, the lecithin containing tetrahydrobiopterin and/or a salt thereof, wherein the composition is stable at room temperature.

42. The article of claim 41, wherein the composition comprises tetrahydrobiopterin.

43. The article of claim 41, wherein the composition comprises a tetrahydrobiopterin salt.

44. (canceled)

45. The article of claim 41, wherein the tetrahydrobiopterin and/or salt thereof is present at at least about 0.5% by weight.

46-47. (canceled)

48. The article of claim 41, wherein the lecithin is present at at least about 0.25% by weight.

49-50. (canceled)

51. The article of claim 41, wherein the lecithin comprises a phosphatidylcholine.

52-54. (canceled)

55. The article of claim 51, wherein at least some of the phosphatidylcholine comprises a polyenylphosphatidylcholine.

56. The article of claim 41, wherein the lecithin is present as a liquid crystal structure.

57-62. (canceled)

63. The article of claim 41, wherein the composition further comprises molecular nitric oxide.

64-65. (canceled)

66. The article of claim 41, wherein the composition is a gel.

67-117. (canceled)
